(19) United States
(12) Patent Application Publication
(10) Pub. No.: US 2023/0038852 A1
(43) Pub. Date: Feb. 9, 2023

(54) AUTOMATED TRACKING SYSTEM

(71) Applicant: Lifetraq Inc., Salt Lake City, UT (US)

(72) Inventor: Erik Kerr, Salt Lake City, UT (US)

(21) Appl. No.: 17/818,223

(22) Filed: Aug. 8, 2022

Related U.S. Application Data
(60) Provisional application No. 63/230,675, filed on Aug. 6, 2021.

(51) Int. Cl. G16H 20/10; G16H 20/70; G16H 20/60

(52) U.S. Cl. CPC G16H 20/10; G16H 20/70; G16H 20/60

(57) ABSTRACT

An automated tracking and analysis system is described. In some instances, the system may collect data describing an attribute of a participant, predict interventions related to the attribute of the participant, and generate personalized tracks relating to the interventions, each of which may include an action. In some instances, the system may assess a performance metric indicating a level of performance of the participant in the personalized tracks, compute a participant score for the personalized tracks using the performance metric; and issue a reward to the participant based on one or more of the participant score and the performance metric.

### BACKGROUND

This application relates to automated tracking and analysis systems. For example, technologies described in this application may use information about patients to exchange and analyze data as well as apply the analysis to improve outcomes for patients.

Currently, people who have previously been incarcerated, have previously had substance abuse problems, and who have mental illness history have a high probability of relapsing. For example, it is very difficult to determine when and who will relapse and individuals are often not helped, especially in a relevant or customized manner. Because people are often lost in large groups of people needing help and insights are not determined for the user, relapsing and recidivism rates are high. Similarly, many business people and otherwise healthy individuals are seeking adaptable and trackable programs that provide accountability and assist them to perform self-improvement tasks.

Unfortunately, disparate systems, data streams, and stakeholders are not integrated, which limits insights and tracking, so participants often fail to advance through tasks that are relevant to them. Existing mentors, parole officers, clinicians, therapists, coaches, and other stakeholders have limited experience and time and therefore cannot detect and predict outcomes for a specific individual due to sparce or noisy data. Accordingly, an automated, intelligent, and interoperable system is desirable.

Additionally, because data is stored and tracked separately, access is not provided to relevant systems and parties, access is not tracked for data-security purposes, and comprehensive solutions are not determined. Accordingly, structures, methods, and systems are desirable to address these data control, access, and analysis issues.

### SUMMARY

An automated tracking and analysis system can be configured to perform particular operations or actions by virtue of having software, firmware, hardware, or a combination of them installed on the system that in operation causes or cause the system to perform the actions. One general aspect of the system includes a method comprising collecting data describing an attribute of a participant; predicting one or more interventions related to the attribute of the participant; generating one or more personalized tracks relating to the one or more interventions, each of the one or more personalized tracks including an action; assessing a performance metric indicating a level of performance of the participant in the one or more personalized tracks; computing a participant score for the one or more personalized tracks using the performance metric; and issuing a reward to the participant based on one or more of the participant score and the performance metric.

Implementations of the system may include one or more of the following features, such as that collecting the data describing the attribute of the participant includes receiving a user input from the participant, the user input describing historical data of the participant, and granting communicative access to receive data from a biometric data-gathering device; that predicting the one or more interventions related to the attribute of the participant includes analyzing the attribute of the participant against a database of other participants to identify a propensity of the participant to relapse respective to the attribute, and determining the one or more interventions based on the propensity of the propensity to relapse and the attribute; that generating the one or more personalized tracks relating to the one or more interventions includes selecting a series of steps relating to the attribute of the participant, the series of steps including the action; and that assessing the performance metric includes determining that completion criteria of the action has been satisfied, determining that a milestone has been completed based on the completion criteria, and apportioning a point value based on the completed milestone, the performance metric including the point value.

Implementations of the operations may include one or more of the following features. That assessing the performance metric includes receiving data from a wearable device, the wearable device tracking biometric data using a biometric sensor, determining that the tracked biometric data exceeds a defined range, and assigning a quantitative value to the performance metric based on the defined range being exceeded; detecting an alert condition based on the performance metric; responsive to detecting the alert condition, issuing an alert to a stakeholder associated with the participant; storing data describing the participant score for the participant score in a centralized management platform, the centralized management platform providing access to the data describing the participant score to one or more third-party servers.

Other implementations of one or more of these aspects include corresponding systems, apparatus, and computer programs, configured to perform the actions of the methods, encoded on computer storage devices.

It should be understood that the language used in the present disclosure has been principally selected for readability and instructional purposes, and not to limit the scope of the subject matter disclosed herein.

### BRIEF DESCRIPTION OF THE DRAWINGS

The disclosure is illustrated by way of example and not by way of limitation in the figures of the accompanying drawings in which like reference numerals are used to refer to similar elements.

FIG. 1 is a block diagram of an example system for providing an automated tracking and analysis tool.

FIG. 2 is a block diagram of an example computing system.

FIG. 3 is a flowchart of an example method for automated tracking and assessment.

FIG. 4 illustrates an example signal diagram providing example interoperability between devices and systems for automated tracking and analysis.

### DESCRIPTION

The present disclosure relates to a system for automated tracking and data analysis. Implementations of the technology provide remote processing and data access controls that provide increased reliability, data security, and reduce locally used processor cycles. In some implementations, the technology provides historical and real-time actionable data and an effective means of disseminating information regarding the progress, risk, and management of an individual at various times. It provides a framework for communicating the severity of specific concerns as well as their likely character and timing.

The technology described herein may provide data integration and interoperability across platforms, which may allow trends to be tracked for individual users and across populations of users, thereby improving insights for individual users in the system. The central and/or remote data control, aggregation, and processing also improve data security, while also allowing improved computer learning, analytics, correlations, and other insights, as described in further detail below.

Aspects of the present disclosure may be applied to tracking and supporting a participant through the process of self-improvement, such as overcoming addiction, a criminal background, a mental health crisis, or other issue while reducing recidivism or relapse. Some aspects of the technology integrate biometric data from a wearable or other biometric device, which may allow automated tracking of data specific to the participant's circumstances, propensities, and issues. A multistep program of challenges, modules, or tasks may be generated for a participant and assessment criteria may be determined and tracked to lead the participant through the program. Some implementations of the technology integrate with various computing systems, such as of health systems, prison systems, rehabilitation centers, etc., to receive and provide access to data, send alerts, or provide rewards to the participant to encourage them through the path of recovery.

For example, in some implementations, the technology provides gamified tracking and accomplishment of tasks to participants, automated tracking and accountability to stakeholders, such as mentors or teams, and rewards. For instance, as a participant advances through a program, badges, ranks, or points may be awarded that encourage users to continue to progress along a program or track. Similarly, reminders and customized tasks, metrics, or interventions may be automatically generated and provided.

For example, the technology may incorporate periodic (e.g., daily, weekly, etc.) tracking by assessment or biometric devices. For instance, a smart wearable device may track steps, activity levels, activity frequency, trend analysis, or hundreds of other data points from users that allow the system or associated stakeholders to provide interventions that help participants who are struggling while also encouraging participants who are succeeding in other areas. The automatically adaptable prescribed tasks and tracking may accordingly help participants to advance at their own pace while predicting potential episodes or relapse.

In some implementations, the technology provides data-driven empowerment that determines the programs or tracks for individuals to provide ongoing support and improve success. For instance, the technology may automatically calibrate based on gathered user data to predict relapse risk, resilience, or advancement.

The technology may be designed to improve program compliance and empower participants to share in the decision-making of their programming outcome design or action plan. With a clear action plan, associated points, and rewards, experts and facilitators can deliver coaching and educational tools to help participants make choices that align with their preferences and values. When individuals participate in their own programming, it improves health and behavior outcomes and reduces the overall cost and complexity of population management significantly. The technology may give participants access to information relating to their condition, remind them of access and actions, provide information for upcoming care and actions, raise alerts or exceptions for their care team's attention, motivate the participants based on a points-based design (e.g., by recognizing and rewarding the participant for progress through a program or track), and build a personalized learning and care journey that matches behavior and propensities of the participant (e.g., to adhere to the program, to take medications, to maintain a diet, to relapse, etc.). The technology is built on behavior economics, gaming science, advanced analytics, and connected devices to increase engagement and support thereby improving outcomes.

The technology described herein provides a dynamic rules engine (e.g., as part of the tracking application 108 or enterprise application 126) that, over time, tailors actions to a participant and their unique circumstances. The system encourages participants to adopt lifestyle behaviors, such as exercising, sleeping regularly, and refraining from smoking, etc. The technology tailors tracking, action plans, and rewards to participants, helps the participants remain accountable, and maintains a communicative connection with experts, such as clinicians, doctors, parole officers, etc., for example, by providing data access to various systems, previously unrelated systems.

For example, the technology may include a centralized management platform that integrates data from survey providers, client devices, biometric sensors, health-care servers, etc. For instance, a central management platform server 122 may receive biometric data (e.g., heart rate variability, sleep patterns, calories, inactivity time, body temperature data, body composition data, etc.,) from a wearable device and use the data to determine progress by the user through milestones or completion metrics of a customized program. A tracking application 108 of the centralized management platform may identify specific markers (e.g., biomarkers, risk factors, actions, etc.) that are relevant to a certain participant's own propensities and attributes. For instance, an inmate with gang-related violence in their past would be automatically tracked differently from a recovering drug addict.

As described in further detail below, the technology may provide historical and real-time actionable data and an effective means of disseminating information regarding the progress, risk, and management of an individual at any time. It provides a framework for communicating the severity of specific concerns as well as their likely character and timing. In some implementations, the technology may provide interoperability, such as the ability of different information technology systems and software applications to communicate, exchange data, and use the information that has been exchanged. It may include data exchange schema and standards that should permit data to be shared across clinician, lab, hospital, pharmacy, and patient computing systems regardless of the application or application vendor, which may permit health information systems to work together within and across organizational boundaries in order to advance the effective delivery of healthcare for individuals and communities. For instance, the platform may provide a database of data that is accessible to various different systems via application programming interfaces or other access means.

In some implementations, the technology may provide interfaces that are unique based on user roles, which may be associated with certain client computing devices, user logins, user profiles, or other data. For instance, an inmate participant may access a unique graphical user interface via a client computing device 106 with limited permissions and restrictions while an administrative role (e.g., of a coach, doctor, or expert) may allow the administrator to view and track participant information, manage connections, etc., via a different or unlocked graphical user interface.

As discussed in further detail below, using these and other technologies, the platform may guide participants and associated stakeholders (e.g., a parole officer, doctor, etc.) through crises, such as depression, sleep disorders, drug addictions, eating disorders, self-harm risk, anger, aggression, violence history. The technology matches the specific crises with modules or steps, metrics, machine learning weights, rewards, and alerts.

The technology may also use gamification to provide points, badges, and rewards to participants. Additionally or alternatively, the metrics may trigger alerts to the associated stakeholders. For instance, various alert levels may be used to identify severity or likelihood of an alert condition (e.g., overdose, suicide, violence, health-crises, etc.), which may be determined based on the collected metrics. In some instances, the metrics may be determined and/or weighted by training a machine learning model trained using a database of other participants data. For example, based on a collected feed of data over time, predictive data sets may become increasingly reliable at detecting patterns, events, threats, etc., based on collected or determined metrics.

These and other features are described in further detail in reference to the figures below. The features and advantages described herein are not all-inclusive and many additional features and advantages are within the scope of the present disclosure. Moreover, it should be noted that the language used in the present disclosure has been principally selected for readability and instructional purposes, and not to limit the scope of the subject matter disclosed herein.

With reference to the figures, reference numbers may be used to refer to example components found in any of the figures, regardless of whether those reference numbers are shown in the figure being described. Further, where a reference number includes a letter referring to one of multiple similar components (e.g., component 000a, 000b, and 000n), the reference number may be used without the letter to refer to one or all of the similar components.

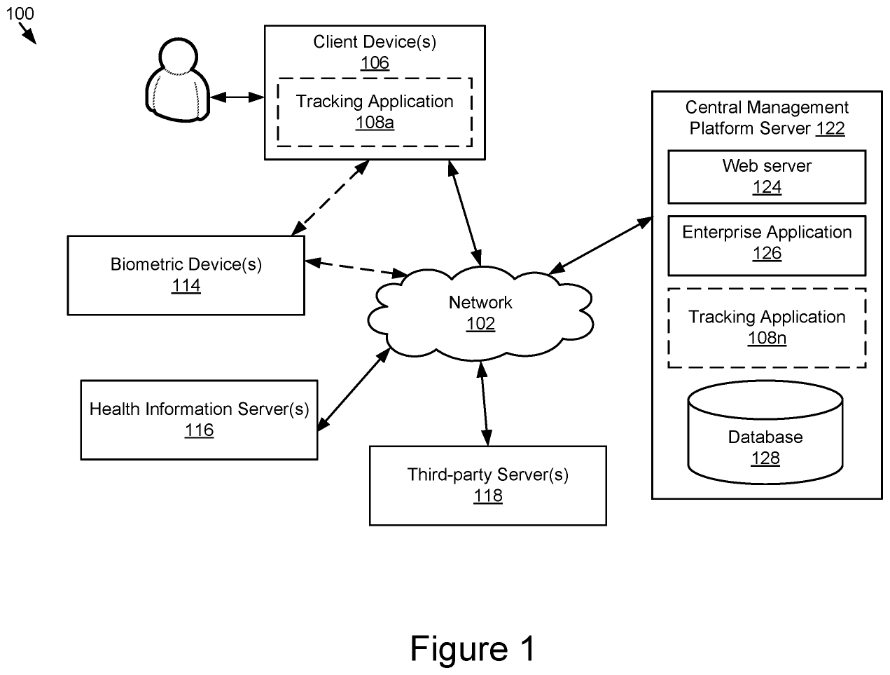

FIG. 1 is a block diagram of an example system 100 for providing an automated tracking tool. The illustrated system 100 may include one or more client devices 106, a third-party server 118, a central management platform server 122, biometric device(s) 114, and/or health information server(s), which may run instances of the tracking application 108a . . . 108n and which may be electronically communicatively coupled via a network 102 for interaction with one another, although other system configurations are possible including other devices, systems, and networks. For example, the system 100 could include any number of client devices 106, biometric devices 114, third-party servers 118, central management platform server(s) 122, and other systems and devices.

The network 102 may include any number of networks and/or network types. For example, the network 102 may include, but is not limited to, one or more local area networks (LANs), wide area networks (WANs) (e.g., the Internet), virtual private networks (VPNs), wireless wide area network (WWANs), WiMAX® networks, personal area networks (PANs) (e.g., Bluetooth® communication networks), various combinations thereof, etc. These private and/or public networks may have any number of configurations and/or topologies, and data may be transmitted via the networks using a variety of different communication protocols including, for example, various Internet layer, transport layer, or application layer protocols. For example, data may be transmitted via the networks using TCP/IP, UDP, TCP, HTTP, HTTPS, DASH, RTSP, RTP, RTCP, VOIP, FTP, WS, WAP, SMS, MMS, XMS, IMAP, SMTP, POP, WebDAV, or other known protocols.

The client device(s) 106 (e.g., multiple client devices 106 may be used by a single participant, multiple participants, stakeholders, or by other users) includes one or more computing devices having data processing and communication capabilities. The client device 106 may couple to and communicate with other client devices 106 and the other entities of the system 100 via the network 102 using a wireless and/or wired connection, such as the central management platform server 122. Examples of client devices 106 may include, but are not limited to, mobile phones, wearables, tablets, laptops, desktops, netbooks, server appliances, servers, virtual machines, TVs, etc. The system 100 may include any number of client devices 106, including client devices 106 of the same or different type.

Although the client devices 106 are illustrated as being separate from biometric devices 114, it should be noted that the client device 106 may include or be directly coupled with biometric devices 114 (or that may serve as a biometric sensor), such as an optical heart rate sensor, electrocardiogram sensor, bioelectrical impedance analysis sensor, accelerometers, microphones, thermometers, hygrometers, or other devices.

The central management platform server 122 and its components may aggregate information about and provide data associated with the systems and processes described herein to a multiplicity of users on a multiplicity of client devices 106, for example, as described in reference to various users and client devices 106 described herein. In some implementations, a single user may use more than one client device 106, which the central management platform server 122 may use to track and aggregate interaction data associated with the user through a variety of different channels including online, physical, and phone (e.g., text, voice, etc.) channels, as discussed elsewhere herein. In some implementations, the central management platform server 122 may communicate with and provide information to a client device 106.

One or more biometric devices 114 may be communicatively coupled with a client device 106 and/or network 102, for example, to collect data about users and provide the data to the tracking application 108 for the operations described herein. For instance, a biometric device 114 may include a device with accelerometers, scales, body composition monitors, breathalyzers, or other sensors, such as an optical heart rate sensor, electrocardiogram sensor, bioelectrical impedance analysis sensor, microphones, thermometers, hygrometers, or other devices. For example, a biometric device 114 may include a smart watch (e.g., an Apple Watch™, Fitbit™, or Samsung Watch Active™), smart ring (e.g., Oura Ring™), smart body composition scale, blood pressure cuff, etc. In some implementations, the biometric device 114 may automatically communicate with an application on a client device 106 to forward data to a tracking application 108, although other implementations are possible and contemplated herein.

In some implementations, the tracking application 108 may send information to another computing device, for example, to a central management platform server 122 or third-party server 118. For example, the tracking application 108 may determine certain information and communicate with various devices based on those details. For instance, the tracking application 108 may perform the various operations described herein.

The central management platform server 122 may include a web server 124, an enterprise application 126, a tracking application 108, and a database 128. In some configurations, the enterprise application 126 and/or tracking application 108 may be distributed over the network 102 on disparate devices in disparate locations or may reside on the same locations, in which case the client device 106a and/or the central management platform server 122 may each include an instance of the tracking application 108 and/or portions thereof. The client devices 106 may also store and/or operate other software such as a tracking application 108, an operating system, other applications, etc., that are configured to interact with the central management platform server 122 via the network 102.

The central management platform server 122 and the third-party server 118 have data processing, storing, and communication capabilities, as discussed elsewhere herein. For example, the servers 122 and/or 118 may include one or more hardware servers, server arrays, storage devices and/or systems, etc. In some implementations, the servers 122 and/or 118 may include one or more virtual servers, which operate in a host server environment.

In some implementations, the enterprise application 126 may receive communications from a client device 106 in order to perform the functionality described herein. The enterprise application 126 may receive information and provide information to the tracking application 108 to generate adaptable graphical interfaces described, as well as perform and provide analytics and other operations. In some implementations, the enterprise application 126 may perform additional operations and communications based on the information received from client devices 106, as described elsewhere herein.

The database 128 may be stored on one or more information sources for storing and providing access to data, such as the data storage device 208. The database 128 may store data describing client devices 106, instances of the tracking application 108, participants, tracks, modules, performance data, scores, etc., such as described herein.

A third-party server 118 can host services such as a third-party application (not shown), which may be individual and/or incorporated into the services provided by the central management platform server 122. For example, the third-party server 118 may represent one or more item databases, forums, company websites, etc. For instance, a third-party server 118 may provide automatically delivered and processed surveys, process biometric data, or perform analytics or other operations. In some instances, the third-party server 118 may represent a computing system of a prison, count health department, rehabilitation system, etc.

The health information server(s) 116 may provide various functionality, such as providing health history for a participant, provide information regarding a condition or participant attribute, etc. In some implementations, the health information server(s) 116 may communicate with the central management platform server 122 to receive participant data, as described in further detail elsewhere herein.

It should be understood that the system 100 illustrated in FIG. 1 is representative of an example system and that a variety of different system environments and configurations are contemplated and are within the scope of the present disclosure. For instance, various acts and/or functionality may be moved from a server to a client, or vice versa, data may be consolidated into a single data store or further segmented into additional data stores, and some implementations may include additional or fewer computing devices, services, and/or networks, and may implement various functionality client or server-side. Further, various entities of the system may be integrated into a single computing device or system or divided into additional computing devices or systems, etc.

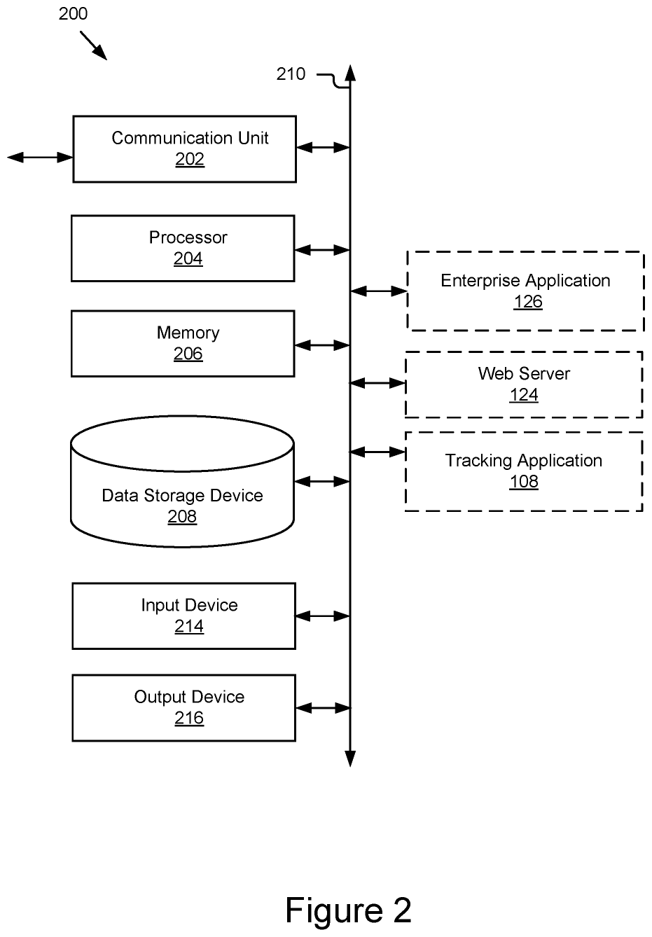

FIG. 2 is a block diagram of an example computing system 200, which may represent computer architecture of a client device 106, third-party server 118, biometric device 114, health information server 116, central management platform server 122, and/or another device described herein, depending on the implementation. In some implementations, as depicted in FIG. 2, the computing system 200 may include an enterprise application 126, a web server 124, a tracking application 108, or another application, depending on the configuration. For instance, a client device 106 may include or execute a tracking application 108 (which could incorporate various aspects of the enterprise application 126, in some implementations); and the central management platform server 122 may include the web server 124, the enterprise application 126, and/or components thereof, although other configurations are also possible and contemplated.

The enterprise application 126 includes computer logic executable by the processor 204 to perform operations discussed elsewhere herein. The enterprise application 126 may be coupled to the data storage device 208 to store, retrieve, and/or manipulate data stored therein and may be coupled to the web server 124, the tracking application 108, and/or other components of the system 100 to exchange information therewith.

The web server 124 includes computer logic executable by the processor 204 to process content requests (e.g., to or from a client device 106). The web server 124 may include an HTTP server, a REST (representational state transfer) service, or other suitable server type. The web server 124 may receive content requests (e.g., product search requests, HTTP requests) from client devices 106, cooperate with the enterprise application 126 to determine the content, retrieve and incorporate data from the data storage device 208, format the content, and provide the content to the client devices 106.

In some instances, the web server 124 may format the content using a web language and provide the content to a corresponding tracking application 108 for processing and/or rendering to the user for display. The web server 124 may be coupled to the data storage device 208 to store retrieve, and/or manipulate data stored therein and may be coupled to the enterprise application 126 to facilitate its operations.

The tracking application 108 includes computer logic executable by the processor 204 on a client device 106 to provide for user interaction, receive user input, present information to the user via a display, and send data to and receive data from the other entities of the system 100 via the network 102. In some implementations, the tracking application 108 may generate and present user interfaces based on information received from the enterprise application 126 and/or the web server 124 via the network 102. For example, a customer/user may use the tracking application 108 to perform the operations described herein. The tracking application 108 may automatically perform the operations and analysis described herein to provide improved automation and tracking.

As depicted, the computing system 200 may include a processor 204, a memory 206, a communication unit 202, an output device 216, an input device 214, and a data storage device 208, which may be communicatively coupled by a communication bus 210. The computing system 200 depicted in FIG. 2 is provided by way of example and it should be understood that it may take other forms and include additional or fewer components without departing from the scope of the present disclosure. For instance, various components of the computing devices may be coupled for communication using a variety of communication protocols and/or technologies including, for instance, communication buses, software communication mechanisms, computer networks, etc. While not shown, the computing system 200 may include various operating systems, sensors, additional processors, and other physical configurations. The processor 204, memory 206, communication unit 202, etc., are representative of one or more of these components.

The processor 204 may execute software instructions by performing various input, logical, and/or mathematical operations. The processor 204 may have various computing architectures to method data signals (e.g., CISC, RISC, etc.). The processor 204 may be physical and/or virtual, and may include a single core or plurality of processing units and/or cores. In some implementations, the processor 204 may be coupled to the memory 206 via the bus 210 to access data and instructions therefrom and store data therein. The bus 210 may couple the processor 204 to the other components of the computing system 200 including, for example, the memory 206, the communication unit 202, the input device 214, the output device 216, and the data storage device 208.

The memory 206 may store and provide access to data to the other components of the computing system 200. The memory 206 may be included in a single computing device or a plurality of computing devices. In some implementations, the memory 206 may store instructions and/or data that may be executed by the processor 204. For example, the memory 206 may store one or more of the enterprise application 126, the web server 124, the tracking application 108, and their respective components, depending on the configuration. The memory 206 is also capable of storing other instructions and data, including, for example, an operating system, hardware drivers, other software applications, databases, etc. The memory 206 may be coupled to the bus 210 for communication with the processor 204 and the other components of computing system 200.

The memory 206 may include a non-transitory computer-usable (e.g., readable, writeable, etc.) medium, which can be any non-transitory apparatus or device that can contain, store, communicate, propagate or transport instructions, data, computer programs, software, code, routines, etc., for processing by or in connection with the processor 204. In some implementations, the memory 206 may include one or more of volatile memory and non-volatile memory (e.g., RAM, ROM, hard disk, optical disk, etc.). It should be understood that the memory 206 may be a single device or may include multiple types of devices and configurations.

The bus 210 can include a communication bus for transferring data between components of a computing device or between computing devices, a network bus system including the network 102 or portions thereof, a processor mesh, a combination thereof, etc. In some implementations, the enterprise application 126, web server 124, tracking application 108, and various other components operating on the computing system/device 100 (operating systems, device drivers, etc.) may cooperate and communicate via a communication mechanism included in or implemented in association with the bus 210. The software communication mechanism can include and/or facilitate, for example, inter-method communication, local function or procedure calls, remote procedure calls, an object broker (e.g., CORBA), direct socket communication (e.g., TCP/IP sockets) among software modules, UDP broadcasts and receipts, HTTP connections, etc. Further, any or all of the communication could be secure (e.g., SSH, HTTPS, etc.).

The communication unit 202 may include one or more interface devices (I/F) for wired and wireless connectivity among the components of the system 100. For instance, the communication unit 202 may include, but is not limited to, various types known connectivity and interface options. The communication unit 202 may be coupled to the other components of the computing system 200 via the bus 210. The communication unit 202 can provide other connections to the network 102 and to other entities of the system 100 using various standard communication protocols.

The input device 214 may include any device for inputting information into the computing system 200. In some implementations, the input device 214 may include one or more peripheral devices. For example, the input device 214 may include a keyboard, a pointing device, microphone, an image/video capture device (e.g., camera), a touch-screen display integrated with the output device 216, etc. The output device 216 may be any device capable of outputting information from the computing system 200. The output device 216 may include one or more of a display (LCD, OLED, etc.), a printer, a haptic device, audio reproduction device, touch-screen display, a remote computing device, etc. In some implementations, the output device is a display which may display electronic images and data output by a processor of the computing system 200 for presentation to a user, such as the processor 204 or another dedicated processor.

In some implementations, the input device 214 may include an integrated or communicatively coupled (e.g., via Bluetooth™) sensor, such as a biometric sensor or device described above.

In some implementations, the communication unit 202 or input device 214 may include various radios or receivers that may be used to receive information, for example, for locating the device 200 (e.g., a client device 106). In some instances, a cellular, Wi-Fi™, or Bluetooth™ radio may be used to determine a location of a client device, for example, by detecting a cell tower, router, or beacon. In some instances, the input device 214 may additionally or alternatively include a GPS sensor for receiving GPS signals and determining a geographic location of the device.

The data storage device 208 may include one or more information sources for storing and providing access to data. In some implementations, the data storage device 208 may store data associated with a database management system (DBMS) operable on the computing system 200. For example, the DBMS could include a structured query language (SQL) DBMS, a NoSQL DMBS, various combinations thereof, etc. In some instances, the DBMS may store data in multi-dimensional tables comprised of rows and columns, and manipulate, e.g., insert, query, update and/or delete, rows of data using programmatic operations.

The data stored by the data storage device 208 may be organized and queried using various criteria including any type of data stored by them, such as described herein. For example, the data storage device 208 may store the database 128. The data storage device 208 may include data tables, databases, or other organized collections of data. Examples of the types of data stored by the data storage device 208 may include, but are not limited to, the data described with respect to the figures, for example, the data may include biometric data, baseline participant data, queries, query responses, user credentials, user roles and permissions, or other data described herein.

The data storage device 208 may be included in the computing system 200 or in another computing system and/or storage system distinct from but coupled to or accessible by the computing system 200. The data storage device 208 can include one or more non-transitory computer-readable mediums for storing the data. In some implementations, the data storage device 208 may be incorporated with the memory 206 or may be distinct therefrom.

The components of the computing system 200 may be communicatively coupled by the bus 210 and/or the processor 204 to one another and/or the other components of the computing system 200. In some implementations, the components may include computer logic (e.g., software logic, hardware logic, etc.) executable by the processor 204 to provide their acts and/or functionality. In any of the foregoing implementations, the components may be adapted for cooperation and communication with the processor 204 and the other components of the computing system 200.

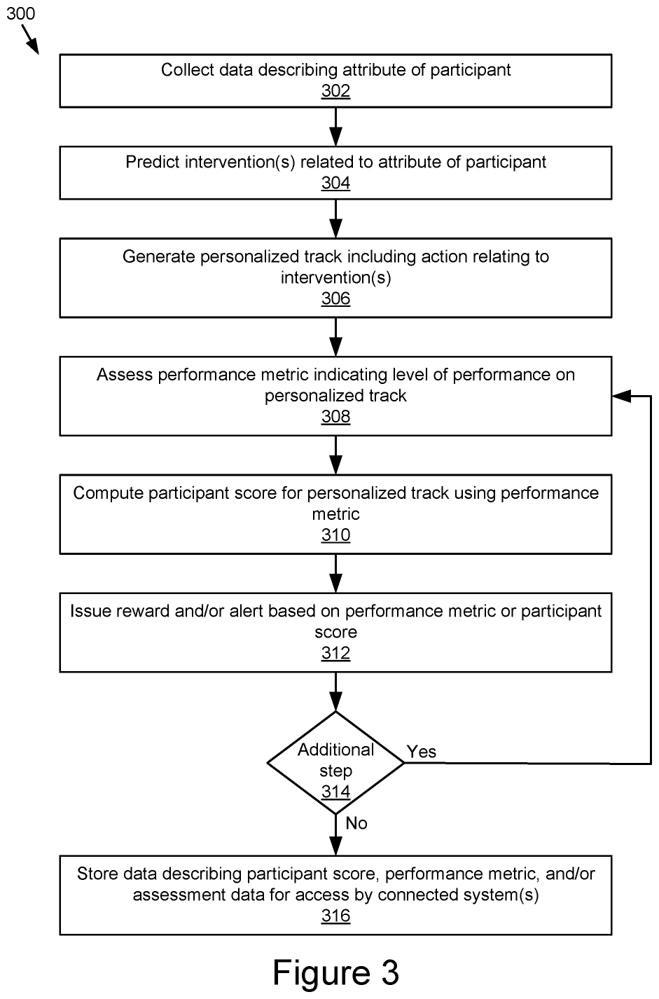

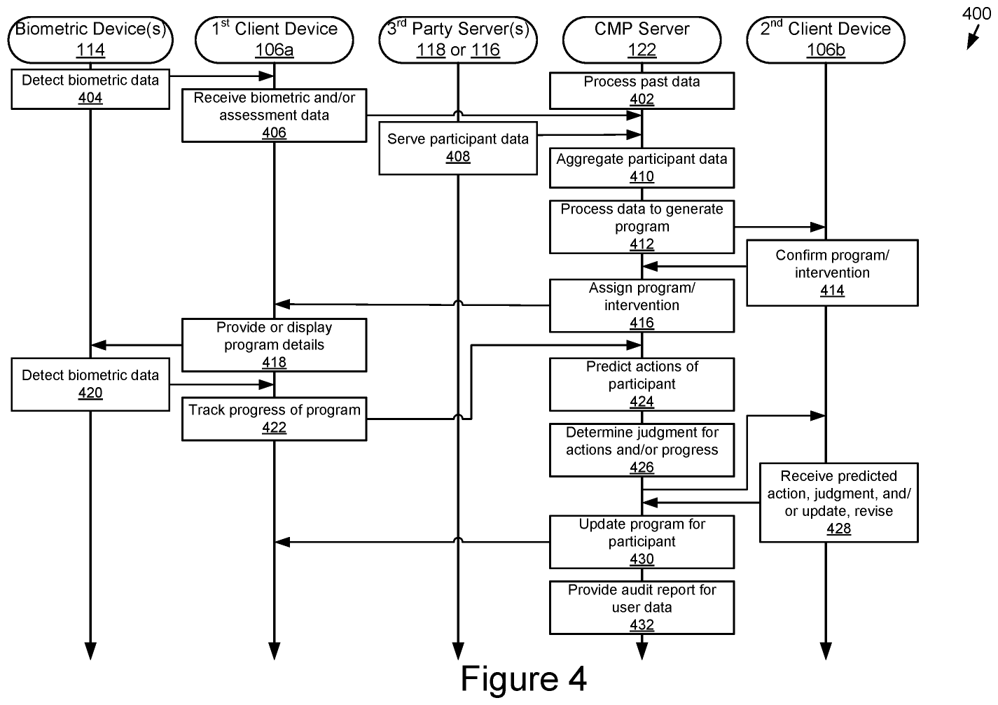

FIG. 3 is a flowchart of an example method 300 for automated tracking and assessment, such as learning metrics for predicting relapse, and other improvements. The method 300 may use technologies described herein, such as the intelligence, analysis, and interoperability between systems described elsewhere herein, such as operations of the diagram 400 described in FIG. 4 and other features described herein.

The provided technology may identify events from a program schedule to enable quantitative measurement of program progress as the program matures over time. For each event, a set of accomplishments identified at the completion of a given stage. Each accomplishment may have an associated set of accomplishment criteria. Construction of the program in this manner enables the stakeholders to collectively measure program progress in an integrated project team environment that is secure and auditable. For example, the tracking application or enterprise application 126 may support conferences and reviews throughout the duration of this contract either in person or via supplied technical input. In some implementations, the system may contribute data and guidance in conferences to facilitate a continuous interchange of ideas, issues, and to identify and resolve potential problem areas. In some implementations, the tracking application may prepare users b providing information and tools as they move through phases of a program or track.

For instance, the method 300 may determine or predict information for a participant (e.g., a certain addiction, a propensity to relapse, other participant data), how the information correlates to existing (or newly or custom programmed) programming and modules, and which markers or metrics to track for the user, program, or module. Accordingly, the tracking application 108 may automatically assess a participant's unique issues and assign them a certain track, set of data, tracking regimen, and associated rewards, alerts, etc. For instance, the tracking application 108 may automatically predict, assess, and assign actions to a user as well as, potentially automated methods (e.g., via biometrics, automatically delivered assessments, or other data) to the participant.

In some implementations, at 302, the tracking application 108 may collect data describing an attribute of a participant.

For example, the tracking application 108 may receive a user input from the participant, which user input describes historical data of the participant. For example, the participant may complete a survey, provide access to health information (e.g., grant permission to medical data from the health information server 116), complete various health assessments (e.g., sleep time, daily assessment of emotions, cravings, lapse status, etc.), or other data. For instance, the participant and/or an associated stakeholder may complete an intake module providing various attributes, conditions, events, propensities, etc., of a participant. Assessments may also be provided to track the participant's progress. For instance, outcome assessments may be delivered based on answers and scores from previous assessments)

In some implementations, the participant may grant communicative access to receive data from a biometric data gathering device 114, such as a smart watch or smart ring. For example, a local instance of the tracking application 108 may be executed on a client device 106 belonging to a participant. The participant may also have a biometric device 114, such as a smart watch, communicatively coupled with the client device 106, which may gather biometric data from the device 114. For example, the participant may instruct the client device 106 to provide access to the biometric data from the biometric device 114 to the tracking application 108. The tracking application 108, depending on the implementation, may communicate with the biometric device 114, application associated therewith (e.g., via an application programming interface) and/or access the biometric data to perform operations, such as determining a baseline assessment, tracking progress through assigned tasks, identifying markers in the data, and predicting relapse or other indicators.

In some implementations, at 304, the tracking application 108 may predict one or more interventions related to the attribute(s) of the participant, for instance, based on the collected data. For example, the tracking application 108 may analyze the attributes of the participant against a database of other participants to identify an attribute (e.g., an addiction), propensity of the participant to relapse respective to the attribute, etc. The tracking application 108 may determine one or more interventions based on the attribute and/or propensity to relapse. For example, an intervention may be a corrective action or goal to accomplish (e.g., overcoming an addiction, etc.). The tracking application 108 may segment or filter a population of participants, predict behavior propensities based on the participant's data and/or that of similar participants (e.g., using a trained model or other previously collected data).

In some implementations, the tracking application 108 may use initial data gathered directly from a participant (e.g., via an assessment), from aggregated biometric data (e.g., over an assessment period), or from other sources, such as other users, a third-party server 118, or a health information server 116. Based on the data, the tracking application 108 may match the user (e.g., based on statistical analysis and/or machine learning) against a database and predict interventions and/or a track of tasks for the participant. For instance, an intervention may include a task for the participant to complete, a set of tasks, a set of data points to collect, or another program.

As an example of the predictive analysis and tracking, which is described in further detail below, the tracking application 108 may predict that the participant has a given propensity to relapse into drug use based on the initial data compared against other users. In some implementation, the tracking application 108 and/or enterprise application 126 may use initial inputs by experts and/or data and outcomes across users to train weights of a supervised machine learning model or otherwise automatically determine statistical correlations. Data may be used to train and update the statistical or machine learning processes to increase reliability over time. As an illustrative example, the tracking application 108 may determine, based on the correlations in the data, that users who have irregular sleeping patterns or above average resting heart rate at night time have an increased likelihood of relapsing. Accordingly, the tracking application 108 may automatically assign a task to the user to improve their bedtime habits. The intervention and/or track may include a series of tasks and tracking measurements for the tasks.

For example, the tracking application 108 may assign an earlier bedtime to the user and then automatically send queries to the user via a client device 106 requesting confirmation that the earlier bedtime is being met or asking the participant to qualitatively or quantitatively indicate whether the earlier bedtime is improving their mental or physical health. In some instances, the queries may be automatically transmitted at defined times. In some implementations, the tracking application 108 may automatically access biometric data and/or request information from a biometric sensor based on the tasks and/or measurements. For instance, the tracking application 108 may automatically access the sensor data to determine a bedtime or heart rate thereby automatically determining whether a task or sub task was completed.

In some implementations, as a participant performs tasks, answers assessments, as additional data is gathered, or as a user moves through a track/program, the intervention, task(s), and/or track may be updated or otherwise automatically adapted.

In some implementations, a medical professional, parole officer, etc., may manually review the participant's information to determine issues, attributes, propensities, and/or interventions, for example, on a second client device and/or account associated with the professional. Accordingly, the automated operations of the tracking application 108 may be overridden or modified by a professional.

For example, at 306, the tracking application 108 may generate one or more personalized programs or tracks including one or more actions relating to the intervention(s). For instance, a particular track may correspond to a given attribute, issue (violence, drug relapse, sleep disorder, etc.), or intervention. A particular track may include one or a series of steps, actions, or challenges to be performed. For example, the tracking application 108 may create highly personalized learning and care journeys that match the behavior propensities of the participant to act (adhere to medications, manage diet, etc.).

In some instances, a track may include a path, series, or set of various predefined steps (e.g., tasks, actions, modules, challenges, etc.) that may be performed by the user. The steps may be sequential or performed in any order, depending on the track or administrative setting. In some implementations, a stakeholder/professional may manually program steps and/or other details of a track or module to be performed by a participant. Tracks or tasks may be tagged for searchability. Tracks or tasks may have defined or definable times, durations, start dates, locations, repeatability, points values, associated batches, associated ranks, or other attributes.

For example, a track, category of track, action, or tracking metric may be based on the specific needs of a participant. A track or action may relate to health, employment, housing, skill development, mentorship, societal networks, etc., and may be tailored based on participant attribute, gender, age, type of crime, type of community, income level, type or severity of addiction, propensity to act or relapse, etc.

Modules or tasks of a track may include an associated metric or completion criteria for evaluating its progress or status. In some implementations, as tasks or tracks are completed, the tracking application 108 may provide informational messages or data, indicate milestones through the steps, etc.

In some implementations, a track may be associated with a group of participants, which may be defined by a stakeholder (e.g., a doctor, counselor, etc.) or automatically generated based on outcomes of previous participants, such as using statistical analysis, clustering, or supervised machine learning.

In some implementations, at 308, the tracking application 108 may assess one or more performance metrics indicating a level of performance on the one or more personalized tracks. For instance, the performance metrics may track a given task or module of a track or the track altogether. For instance, the tracking application 108 may determine whether assessed or biometric data exceeds or otherwise satisfies a determined threshold or range. The threshold may be expressly defined or may be determined based on machine learning, statistical analysis, or other processing, as described elsewhere herein.

In some implementations, the tracking application 108 may determine that a completion criterion of the action has been satisfied (e.g., based on a threshold being satisfied). For example, if the task includes learning a skill, the completion criterion may include a certification of the skill. The tracking application 108 may determine that a milestone has been completed and/or apportion a point value based on the completed criterion and/or the milestone (e.g., if the milestone represents multiple tasks).

In some implementations, a completion criterion may require proof or validation. For instance, the tracking application 108 may allow a participant (e.g., via a client device) to upload a video, photo, survey response, etc., to prove completion of a task/module. In some implementations, the tracking application 108 may automatically track data patterns (e.g., of metrics, survey data, upload patterns, etc.) and allow the data to be audited or automatically detect suspicious patterns.

In some implementations, a metric associated with a track, module, etc., may be based on biometric data, which may be manually or automatically input into the tracking application 108. For instance, a biometric device 114 may automatically track sleep or exercise regularity, heart rate variability, breathing rate, body temperature, other sleep metrics, calories burned, biometric device 114 reporting data, etc. For example, the tracking application 108 may receive data from a wearable device and determine that tracked biometric patterns exceed/satisfy a defined range or threshold. The tracking application 108 may assign a quantitative value to a performance metric based on the range or threshold being exceeded/satisfied.

In some implementations, the metric may be determined based on or including survey data. For instance, a third-party server 118 or central management platform server 122 may automatically (e.g., at a defined time, task, or trigger) send an assessment survey requesting quantitative and/or qualitative input from the participant. For instance, an assessment may include self-reported measures, such as mental state, behavioral patterns, health data, quality of life, addiction severity, withdrawal, emotions, cravings, or other data. Various inputs may be weighted or otherwise associated with scores, may be binary to indicate completion, or may automatically trigger rewards or alerts.

The assessment and/or metrics may be based on historical data received from the participant to track the participant's progress accurately across time. The tracking application 108 may additionally or alternatively evaluate a track, program, or module to determine its effectiveness for the participant, so that the program may be adapted to better suit the participant.

In some implementations, as a participant works through steps of a track, the tracking application 108 may provide information or counseling. For example, via an administrative graphical user interface, a professional/stakeholder may add articles, videos, podcasts, or other media that provides information relating to collected data, analytics, predicted analytics, tasks, or otherwise. In some implementations, instances of the tracking application 108 and or the enterprise application 126 may provide direct communication between participants and professionals or automatedly schedule meetings.

In some implementations, at 310, the tracking application 108 may compute a participant score for the participant and the one or more personalized tracks using the performance metrics. For instance, the tracking application 108 may determine an overall score for the participant, for the track, for a module/task, or a category of tracks or modules. For example, the tracking application 108 may compute an overall score for the participant to indicate their progress. In another example, the tracking application 108 may compute a score representing a level of skill of the user, for example, for completing modules in a category relating to that skill.

In some implementations, at 312, the tracking application 108 may issue a reward and/or an alert based on the performance metric or the participant score. For example, the tracking application 108 may detect that a milestone or threshold score has been reached and issue an award or reward based on the milestone. For example, in order to motivate the participant, the tracking application 108 may provide a level up, badge, token, status level, reward point(s) or other reward. The status may provide a right/privilege for an inmate or recently released inmate. In some instances, a monetary or other reward may be provided to support recovery at defined milestones. In some instances, the tracking application 108 may automatically customize a reward to a participant, for example, based on the milestone or data collected (e.g., at 102).

In some implementations, the tracking application 108 may detect an alert condition based on a performance metric, score, or other collected data. For instance, if a biometric device 114 (e.g., via a tracking application 108) or assessment detects a defined or determined condition or fails to report (e.g., if a biometric device 114 is deactivated or a participant fails to respond to a query), an alert may be triggered. Similarly, a failure to meet a milestone, a survey response, or another trigger may cause the tracking application 108 to automatically issue an alert to a stakeholder associated with the participant or to the participant.

In some implementations, the tracking application 108 may compute a threat level indicating a severity and/or a likelihood of an action (e.g., relapse) based on the performance metric(s) or other data collected. For instance, if failure to complete a task or milestone, biometric data, and survey data are detected together, a severity or likelihood of relapse may be greater than if one of these conditions is detected separately. Accordingly, the tracking application 108 may determine a threat level, which may correspond to a score or color.

In some instances, an alert may be issued to the participant, an associated stakeholder, or future tasks or tracks may be adjusted or generated based on the alert.

In some implementations, at 314, the tracking application 108 may determine whether there is another step in the personalized track or tracks. For instance, the tracking application 108 may iteratively process tasks/modules and/or tracks. Similarly, as noted above, as data is collected, a track and associated actions/tasks may be dynamically updated and customized for a user.

In some implementations, at 316, the tracking application 108 may store data describing the participant score, performance metric, and/or assessment data for access by one or more connected system(s). For instance, the tracking application 108 may store the data in a database 128 of a central management platform server 122, which may, in turn, provide access to the data, metrics, rewards, badges, etc., to other systems, such as the client device(s) 106, health information server(s) 116, or third-party server(s) 118.

The database 128 may be designed to enforce referential integrity, primary or foreign keys, unique indexes, check constraints, and triggers. Further, the database 128 may provide for the generation and use of audit logs to detect data patterns, suspicious information, anomalies, etc. For instance, the central management platform server 122 may provide audit logs indicating when data was accessed and by whom/what device, possible breaches, or other information. Accordingly, using the logs, suspicious patterns of activity can be identified.

Accordingly, the data associated with the participant may be carried forward to future programs, tracks, or systems. For instance, aggregated data and analytics for a user may be associated with that user for a future program and/or transmitted in a standard data format to another system. For example, a participant may move on to a new program and/or be associated with a new stakeholder, and access to the data may automatically be transferred by the enterprise application 126 and/or tracking application 108 to a new stakeholder/professional.

FIG. 4 illustrates an example signal diagram 400 providing example interoperability between devices and systems and automated tracking and analysis. For example, operations and communications of the diagram may be used in conjunction with, in addition to, or alternative to other operations described herein, such as in the method 300 described in reference to FIG. 3. For example, operations of the diagram 400 may augment and/or enable operations of the method 300 of FIG. 3. It should be noted that the example signal diagram 400 is illustrated in reference certain components of the system 100, although other implementations are possible. In some instances, operations of the diagram 400 are provided using hardware and/or logic of the respective devices. For instance, instances or components of the tracking application 108, web server 124, enterprise application 126, etc., may be executed separately or in a distributed manner to facilitate the operations described herein.

It should further be noted that the operations and features of the figures herein, including those of FIG. 4, are provided by way of example and that other, fewer, or additional operations and features may be used without departing from the scope of this disclosure. For example, some tasks of a track or program may be tracked automatically using a biometric device 114 while other tasks may be tracked using quantitative or qualitative (e.g., converted by a tracking application 108 or enterprise application 126 to numerical quantities for processing) assessments, which may be surveys with questions to which a participant responds. Similarly, operations of the diagram 400 may be performed recursively or multiple times to update or adjust the data, tasks, or other features for a user or group of users. Similarly, the operations may be performed in a different order or in parallel instead of or in addition to linearly as in the provided example implementation.

Interoperability of different information technology systems and software applications allows the systems to communicate, exchange data, and use the information that has been exchanged. Defined data exchange schema and standards may be used to permit data to be shared by clinician, lab, hospital, pharmacy, participant, or stakeholder, etc., regardless of application or application vender. For instance, this allows systems to function together, for example, across organizational boundaries in order advance effective processing and interchange of data.

In some implementations, at 402, the central management platform server 122 may process past data to determine weights, analytics, statistics, correlations, programs, tasks, or other data. For instance, participants, administrators, or professionals may input data about individuals, tasks, and outcomes either individually or as a batch. In some implementations, the professionals or participants may input tasks, task-completion metrics or tracking methods, tracks, programs, or other defined data, as described herein.

In some implementations, the central management platform server 122 may feed the participant data, such as biometric and survey data into a supervised machine learning algorithm along with outcomes for the participants to train models and weights and identify correlations. It should be noted that various computer learning algorithms, models, or statistical methods may be used to analyze the data.

In some implementations, a participant may begin a program by creating a profile or account with the central management platform server 122 and/or downloading a tracking application 108 to the participant's client device 106a. The participant may be invited by a professional or account associated with an organization (e.g., a parole officer, hospital, school, etc.) and the tracking application 108 may automatically link their accounts and data.

As described elsewhere herein, the central management platform server 112 may provide data access only to authorized individuals and computing systems, track data access, and ensure compliance with privacy and data protection laws and policies, such as HIPAA (Health Insurance Portability and Accountability Act of 1996). For instance, only authenticated, logged-in users may access the participant's data, which may be kept only on a database 128 of the central management platform server 122 or other secure data-access computing device, which tracks, secures (e.g., by encryption), and limits access to computing devices.

In some implementations, a biometric device 114 may detect biometric data at 404, which it may transmit to a first client device 106a of a participant. The participant may allow access to the biometric data on the first client device 106a and the tracking application 108 may use custom or built-in (e.g., in the operating system of the client device 106) APIs to access the biometric device 114 or data. For instance, the biometric device 114 may send biometric data to the first client device 106a via a Bluetooth™ connection. The first client device 106a may provide access to the tracking application 108 to the biometric data and/or communication or control of the biometric device 114. The first client device 106a may process the data and/or relay it to a central management platform server 122 to process it.

In some implementations, the tracking application 108, via the first client device 106a may serve a questionnaire or assessment to the participant, who may enter data, as described herein. At 406, the first client device 106a may receive the biometric and/or assessment data and transmit it to the central management platform server 122, for example, via an encrypted data transmission via the network 102, as described elsewhere herein.

In some implementations, the central management platform server 122 may retrieve data from a third-party server 118 and/or health information server 116 to further inform the participant's profile and improve analysis at 408. For example, the central management platform server 122 may, at 410, aggregate data for the participant using biometric data, assessment data, or data from other servers 116 or 118.

At 412, the central management platform server 122 may process the data to generate a program of tasks, tracks, metrics, assessments, interventions, etc., as described elsewhere herein. In some implementations, in order to securely and privately process the data, the analysis may be performed behind a firewall and/or on a single system.

Accordingly, the data may be integrated and processed on a secure and compliant computing system. Additionally, because the data may be integrated across devices but still processed on a secure system, trends may be better tracked over time for individual or groups of participants. It should be noted that it is very difficult or impossible to perform reliable analysis across large numbers of participants, tasks, and outcomes by a human using manual processes. The central management platform server 122, on the other hand, may process large datasets having less reliable or delayed feedback, thereby detecting correlations and insights across a sparce and/or noisy dataset in a way that is not possible manually. Additionally, the central management platform server 122 may securely process and provide data access in a way that is enabled by a secure computing system.

In some implementations, at 414, the central management platform server 122 may transmit processed data, predicted outcomes, and/or generated programs or interventions to a second client device 106b of a professional/stakeholder. For instance, the stakeholder may view the data via the second client device 106b (e.g., either via tracking application 108 or a web-interface provided by the web server 124) and provided by the central management platform server 122, which access may be securely provided to the specific stakeholder based on the stakeholder's role, linked account, and/or user credentials. In some implementations, the stakeholder may confirm or modify the program, tasks, tracking metrics, assessments, interventions, or participant data.

In some implementations, at 416, the central management platform server 122 may assign the program or intervention to the participant based on the received participant data, processed data, predictions, and/or stakeholder modifications, as described in further detail above. The central management platform server 122 may transmit the program and associated tasks, tracks, assessments, metrics, etc., to the tracking application 108 on the first client device 106a or otherwise provide access to the data through pushed, pulled, batched, periodically, or continuously provided data communication. The participant may view the assigned information via the first client device 106a and/or another computing device authenticated with the central management platform server 122, for example, at 418, the first client device 106a may display program details on a graphical user interface on an output device of the device 106a.

In some implementations, the tasks and/or tracking metrics may include biometric data and the tracking application 108 may trigger collection or access of the data via the first client computing device 106a and/or a biometric device 114 at 420, as described above.

In some implementations, the tasks and/or tracking metrics may include other inputs, such as responses to questions or other assessments by the user. For example, at a defined time (e.g., after a task's completion date or periodically), the tracking application 108 may provide a question or otherwise request input from the participant to an assessment via the first client device 106a.

At 422, the progress of the program, tasks, and/or metrics may be tracked using the assessment responses, biometric data, and/or other data. The tracking may be performed on the first client device 106a, the central management platform server 122, another device, or a combination thereof. For example, the first client device 106a may automatically transmit the biometric, assessment, or other data to the central management platform server 122, which processes the data.

As described in further detail above, the central management platform server 122 may process the data to predict actions of the participant at 424, such as a probability of recidivism or relapse. For instance, the enterprise application 126 may match the tracked data against defined tracks/programs to determine a subsequent step. For instance, if a first task was completed or a certain input was received, the tracking application 108 or enterprise application 126 may use a decision tree (e.g., programmed by a stakeholder or administrator) to determine a subsequent task, metric, or reward for the participant. In some implementations, the tracking application 108 or enterprise application 126 may match the participant's data against the determined correlations and/or apply trained machine learning model(s) to determine a likelihood of an action (e.g., relapse) or track a participant's progress.

For example, at 426, the central management platform server 122 may use a decision tree, machine learning model, or statistical analysis to determine whether a user has completed a step of a track or program and assign a next step or reward, or perform another operation as described in further detail above.

In some implementations, the central management platform server 122 may transmit a notification, provide access, or otherwise provide information to a stakeholder associated with the participant in the system. For instance, the second client device 106b may receive or access (e.g., via a local instance of a graphical user interface or a web interface) a predicted action, judgment, or other data at 428. The stakeholder may also manually enter or revise the predicted action, judgement, reward, or other data. Accordingly, the program and rewards can be tailored to the participant and his/her own progress to improve reliability, communication, and outcomes.

In some implementations, additional stakeholders may be included in data access and communication for a participant, for example, a first stakeholder or participant may provide access to a second stakeholder, such as a therapist. For instance, a first stakeholder and/or participant may instruct the enterprise application 126 to provide data access to a user account associated with a second stakeholder.

In some implementations, as data is gathered from biometric device(s) 113 or assessments, deviations from baselines or satisfaction of defined criteria may trigger an alert, which may be sent to a stakeholder or support person. For example, changes to a baseline sleep schedule, heart rate, calorie intake, or other triggers may cause the technology to automatically notify a stakeholder about possible deterioration of the participant's state and allow the stakeholder to offer additional support to the participant at a time when the support may be most beneficial. Similarly, if biometric data or assessment responses are not received, the stakeholder may be notified, or the information may be used to predict participant actions or outcomes.

In some implementations, at 430 the updated program data and/or reward data may be provided the participant, for example, by transmitting it to the first client device 106a. Accordingly, a participant's progress may be tracked over time, a track or program may be automatically adapted to the participant and their progress, and outcomes can be positively influenced either automatically or by providing analytics to stakeholders.

In some implementations, the tracking application 108, third-party server 118, and/or central management platform server 122 may facilitate redemption of rewards by the participant.

In some implementations, at 432, the central management platform server 122 may generate and provide an audit report for user data, such as health data, participant data, etc., which is stored or processed by the central management platform server 122. For instance, the audit report may indicate which data is stored, access details (by whom, when, which devices, etc.) for the data, data-use patterns, or other data-security and privacy information, although other implementations are possible and contemplated herein.

In the above description, for purposes of explanation, numerous specific details are set forth in order to provide a thorough understanding of the present disclosure. However, it should be understood that the technology described herein can be practiced without these specific details. Further, various systems, devices, and structures are shown in block diagram form in order to avoid obscuring the description. For instance, various implementations are described as having particular hardware, software, and user interfaces. However, the present disclosure applies to any type of computing device that can receive data and commands, and to any peripheral devices providing services.

In some instances, various implementations may be presented herein in terms of algorithms and symbolic representations of operations on data bits within a computer memory. An algorithm is here, and generally, conceived to be a self-consistent set of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

To ease description, some elements of the system 100 and/or the methods are referred to using the labels first, second, third, etc. These labels are intended to help to distinguish the elements but do not necessarily imply any particular order or ranking unless indicated otherwise.

It should be borne in mind, however, that all of these and similar terms are to be associated with the appropriate physical quantities and are merely convenient labels applied to these quantities. Unless specifically stated otherwise as apparent from the following discussion, it is appreciated that throughout this disclosure, discussions utilizing terms including “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Various implementations described herein may relate to an apparatus for performing the operations herein. This apparatus may be specially constructed for the required purposes, or it may comprise a general-purpose computer selectively activated or reconfigured by a computer program stored in the computer. Such a computer program may be stored in a computer readable storage medium, including, but is not limited to, any type of disk including floppy disks, optical disks, CD-ROMs, and magnetic disks, read-only memories (ROMs), random access memories (RAMs), EPROMs, EEPROMs, magnetic or optical cards, flash memories including USB keys with non-volatile memory or any type of media suitable for storing electronic instructions, each coupled to a computer system bus.

The technology described herein can take the form of an entirely hardware implementation, an entirely software implementation, or implementations containing both hardware and software elements. For instance, the technology may be implemented in software, which includes but is not limited to firmware, resident software, microcode, etc. Furthermore, the technology can take the form of a computer program product accessible from a computer-usable or computer-readable medium providing program code for use by or in connection with a computer or any instruction execution system. For the purposes of this description, a computer-usable or computer readable medium can be any non-transitory storage apparatus that can contain, store, communicate, propagate, or transport the program for use by or in connection with the instruction execution system, apparatus, or device.

A data processing system suitable for storing and/or executing program code may include at least one processor coupled directly or indirectly to memory elements through a system bus. The memory elements can include local memory employed during actual execution of the program code, bulk storage, and cache memories that provide temporary storage of at least some program code in order to reduce the number of times code must be retrieved from bulk storage during execution. Input or I/O devices (including but not limited to keyboards, displays, pointing devices, etc.) can be coupled to the system either directly or through intervening I/O controllers.

Network adapters may also be coupled to the system to enable the data processing system to become coupled to other data processing systems, storage devices, remote printers, etc., through intervening private and/or public networks. Wireless (e.g., Wi-Fi™) transceivers, Ethernet adapters, and Modems, are just a few examples of network adapters. The private and public networks may have any number of configurations and/or topologies. Data may be transmitted between these devices via the networks using a variety of different communication protocols including, for example, various Internet layer, transport layer, or application layer protocols. For example, data may be transmitted via the networks using transmission control protocol/Internet protocol (TCP/IP), user datagram protocol (UDP), transmission control protocol (TCP), hypertext transfer protocol (HTTP), secure hypertext transfer protocol (HTTPS), dynamic adaptive streaming over HTTP (DASH), real-time streaming protocol (RTSP), real-time transport protocol (RTP) and the real-time transport control protocol (RTCP), voice over Internet protocol (VOIP), file transfer protocol (FTP), WebSocket (WS), wireless access protocol (WAP), various messaging protocols (SMS, MMS, XMS, IMAP, SMTP, POP, WebDAV, etc.), or other known protocols.

Finally, the structure, algorithms, and/or interfaces presented herein are not inherently related to any particular computer or other apparatus. Various general-purpose systems may be used with programs in accordance with the teachings herein, or it may prove convenient to construct more specialized apparatus to perform the required method blocks. The required structure for a variety of these systems will appear from the description above. In addition, the specification is not described with reference to any particular programming language. It will be appreciated that a variety of programming languages may be used to implement the teachings of the specification as described herein.

The foregoing description has been presented for the purposes of illustration and description. It is not intended to be exhaustive or to limit the specification to the precise form disclosed. Many modifications and variations are possible in light of the above teaching. As will be understood by those familiar with the art, the specification may be embodied in other specific forms without departing from the spirit or essential characteristics thereof. Likewise, the particular naming and division of the modules, routines, features, attributes, methodologies, and other aspects are not mandatory or significant, and the mechanisms that implement the specification or its features may have different names, divisions and/or formats. Furthermore, the modules, routines, features, attributes, methodologies, and other aspects of the disclosure can be implemented as software, hardware, firmware, or any combination of the foregoing. Also, wherever a component, an example of which is a module, of the specification is implemented as software, the component can be implemented as a standalone program, as part of a larger program, as a plurality of separate programs, as a statically or dynamically linked library, as a kernel loadable module, as a device driver, and/or in every and any other way known now or in the future. Additionally, the disclosure is in no way limited to implementation in any specific programming language, or for any specific operating system or environment.

What is claimed is:

1. A computer-implemented method comprising:
collecting, by a processor, data describing an attribute of a participant;
predicting, by the processor, one or more interventions related to the attribute of the participant;
generating, by the processor, one or more personalized tracks relating to the one or more interventions, each of the one or more personalized tracks including an action;
assessing, by the processor, a performance metric indicating a level of performance of the participant in the one or more personalized tracks;
computing, by the processor, a participant score for the one or more personalized tracks using the performance metric; and
issuing, by the processor, a reward to the participant based on one or more of the participant score and the performance metric.

2. The computer-implemented method of claim 1, wherein collecting the data describing the attribute of the participant includes:
receiving a user input from the participant, the user input describing historical data of the participant; and
granting communicative access to receive data from a biometric data-gathering device.

3. The computer-implemented method of claim 1, wherein predicting the one or more interventions related to the attribute of the participant includes:
analyzing the attribute of the participant against a database of other participants to identify a propensity of the participant to relapse respective to the attribute; and
determining the one or more interventions based on the propensity of the propensity to relapse and the attribute.

4. The computer-implemented method of claim 1, wherein generating the one or more personalized tracks relating to the one or more interventions includes:
selecting a series of steps relating to the attribute of the participant, the series of steps including the action.

5. The computer-implemented method of claim 1, wherein assessing the performance metric includes:
determining that completion criteria of the action has been satisfied;
determining that a milestone has been completed based on the completion criteria; and
apportioning a point value based on the completed milestone, the performance metric including the point value.

6. The computer-implemented method of claim 1, wherein assessing the performance metric includes:
receiving data from a wearable device, the wearable device tracking biometric data using a biometric sensor;
determining that the tracked biometric data exceeds a defined range; and
assigning a quantitative value to the performance metric based on the defined range being exceeded.

7. The computer-implemented method of claim 1, further comprising:
detecting, by the processor, an alert condition based on the performance metric; and
responsive to detecting the alert condition, issuing, by the processor, an alert to a stakeholder associated with the participant.

8. The computer-implemented method of claim 1, further comprising:
storing, by the processor, data describing the participant score for the participant score in a centralized management platform, the centralized management platform providing access to the data describing the participant score to one or more third-party servers.

9. A system comprising:
one or more processors; and
a computer memory storing instructions that, when executed by the one or more processors, cause the system to perform operations comprising:
collecting data describing an attribute of a participant;
predicting one or more interventions related to the attribute of the participant;
generating one or more personalized tracks relating to the one or more interventions, each of the one or more personalized tracks including an action;
assessing a performance metric indicating a level of performance of the participant in the one or more personalized tracks;
computing a participant score for the one or more personalized tracks using the performance metric; and
issuing a reward to the participant based on one or more of the participant score and the performance metric.

10. The system of claim 9, wherein collecting the data describing the attribute of the participant includes:
receiving a user input from the participant, the user input describing historical data of the participant; and
granting communicative access to receive data from a biometric data-gathering device.

11. The system of claim 9, wherein predicting the one or more interventions related to the attribute of the participant includes:
analyzing the attribute of the participant against a database of other participants to identify a propensity of the participant to relapse respective to the attribute; and
determining the one or more interventions based on the propensity of the propensity to relapse and the attribute.

12. The system of claim 9, wherein generating the one or more personalized tracks relating to the one or more interventions includes:
selecting a series of steps relating to the attribute of the participant, the series of steps including the action.

13. The system of claim 9, wherein assessing the performance metric includes:
determining that completion criteria of the action has been satisfied;
determining that a milestone has been completed based on the completion criteria; and
apportioning a point value based on the completed milestone, the performance metric including the point value.

14. The system of claim 9, wherein assessing the performance metric includes:
receiving data from a wearable device, the wearable device tracking biometric data using a biometric sensor;
determining that the tracked biometric data exceeds a defined range; and
assigning a quantitative value to the performance metric based on the defined range being exceeded.

15. The system of claim 9, wherein the operations further comprise:
detecting an alert condition based on the performance metric; and
responsive to detecting the alert condition, issuing an alert to a stakeholder associated with the participant.

16. The system of claim 9, wherein the operations further comprise:
storing data describing the participant score for the participant score in a centralized management platform, the centralized management platform providing access to the data describing the participant score to one or more third-party servers.

17. A non-transitory computer-readable medium storing instructions that, when executed by a computer, cause the computer to perform operations comprising:
collecting data describing an attribute of a participant;
predicting one or more interventions related to the attribute of the participant;
generating one or more personalized tracks relating to the one or more interventions, each of the one or more personalized tracks including an action;
assessing a performance metric indicating a level of performance of the participant in the one or more personalized tracks;
computing a participant score for the one or more personalized tracks using the performance metric; and
issuing a reward to the participant based on one or more of the participant score and the performance metric.

18. The non-transitory computer-readable medium of claim 17, wherein collecting the data describing the attribute of the participant includes:
receiving a user input from the participant, the user input describing historical data of the participant; and
granting communicative access to receive data from a biometric data-gathering device.

19. The non-transitory computer-readable medium of claim 17, wherein predicting the one or more interventions related to the attribute of the participant includes:
analyzing the attribute of the participant against a database of other participants to identify a propensity of the participant to relapse respective to the attribute; and
determining the one or more interventions based on the propensity of the propensity to relapse and the attribute.

20. The non-transitory computer-readable medium of claim 17, wherein generating the one or more personalized tracks relating to the one or more interventions includes:
selecting a series of steps relating to the attribute of the participant, the series of steps including the action.
